(19) United States
(12) Patent Application Publication
(10) Pub. No.: US 2022/0356484 A1
(43) Pub. Date: Nov. 10, 2022

(54) GENETIC MODIFICATION OF PLANTS

(71) Applicant: Dr. James Berman, Merion Station, PA (US)

(72) Inventor: James Berman, Merion Station, PA (US)

(21) Appl. No.: 17/640,634

(22) PCT Filed: Sep. 3, 2020
(86) PCT No.: PCT/US2020/049102

§ 371 Date: Mar. 4, 2022

Related U.S. Application Data
(60) Provisional application No. 62/896,737, filed on Sep. 6, 2019.
(60) Provisional application No. 62/939,077, filed on Nov. 22, 2019.

(51) Int. Cl. C12N 15/82

(52) U.S. Cl. CPC C12N 15/8213; C12N 15/8243

(57) ABSTRACT

Gene editing complexes are specifically directed to cannabinoid sequences, such as tetrahydrocannabinol (THC), for excision or inactivation of these sequences. The disclosure is directed to the inhibition of synthesis of THC in a cannabis plant. In doing so, THC would never become an active compound within the plant chemistry and chemotype, thereby eliminating the chance of CBD extracts being contaminated with THC.

### CROSS REFERENCE TO RELATED APPLICATIONS

This Application claims the benefit of U.S. Provisional Application 62/939,077 filed on Nov. 22, 2019 and U.S. Provisional Application 62/896,737 filed on Sep. 6, 2019. The entire contents of these applications are incorporated herein by reference in their entirety.

### FIELD OF THE DISCLOSURE

The present disclosure relates in general to methods of gene editing and gene excision in plants. The disclosure relates in particular to inactivating or excision of, for example, tetrahydrocannabinol (THC) or modulation of expression of particular cannabinoids.

### BACKGROUND

The hemp industry is rapidly evolving, particularly with the purported medicinal qualities of cannabidiol (CBD and tetrahydrocannabinol (THC). Despite the myriad claims of proven efficacy, or even cure, for a laundry list of medical maladies, peer reviewed literature provides scant corroboration of such claims. Moreover, there is ample scientific evidence that tetrahydrocannabinol (THC), the psychoactive compound found within Cannabis Sativa L, is deleterious to the growing adolescent brain, may lead to dependency, and may have harmful effects, such as neurocognitive dysfunction and sinopulmonary complications.

Moreover, differentiating cannabidiol (CBD) from tetrahydrocannabinol (THC), as it pertains to percent composition of a marketed nutraceutical product, is suspect at best, as true laboratory standardization essentially does not exist to date.

### SUMMARY

The disclosure is directed to the inhibition of synthesis of THC in a cannabis plant. In doing so, THC would never become an active compound within the plant chemistry and chemotype, thereby eliminating the chance of CBD extracts being contaminated with THC.

In certain embodiments, cannabis plants are contacted with gene editing agents which specifically excise the THC gene or inactivate the expression of the THC gene.

In certain embodiments, gene editing complexes are directed to gene regulatory regions of cannabinoids, e.g., cannabidiol (CBD) to enhance the expression of desired cannabinoids.

In certain embodiments, gene editing complexes are directed to gene regulatory regions of cannabinoids, e.g., cannabidiol (CBD) and enhance the expression of desired cannabinoids and/or decrease the expression of undesired cannabinoids.

In certain embodiments, cannabis plants are contacted with (i) gene editing agents which specifically excise the THC gene or inactivate the expression of the THC gene and (ii) gene editing complexes which are directed to gene regulatory regions of cannabinoids, e.g., cannabidiol (CBD) to enhance the expression of desired cannabinoids and/or (iii) expression vectors which express cannabinoid genes.

In certain embodiments, the gene-editing agents comprise: CRISPR/Cas systems. Examples include Cas9, spCas9-NG, base editing, xCas9, Cpf1, Cas13, Cas14 and the like.

Other aspects are described infra.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, “base editing” (BE) is a genome editing system that introduces precise and highly predictable nucleotide changes at genomic targets without requiring donor DNA templates or double-stranded breaks (DSBs) and are not dependent on homology-directed repair (HDHDR) and non-homologous end-joining (NHEJ).

As used herein, the term “cannabinoid” means any substance that acts upon a cannabinoid receptor. For example, the term cannabinoid includes cannabinoid ligands such as agonists, partial agonists, inverse agonists, or antagonists, as demonstrated by binding studies and functional assays. In many examples, a cannabinoid can be identified because its chemical name will include the text string “*cannabi*” in the name. Within the context of this application, where reference is made to a particular cannabinoid, each of the acid and/or decarboxylated forms are contemplated as both single molecules and mixtures. Examples of cannabinoids within the context of this disclosure include compounds belonging to any of the following classes of molecules, their derivatives, salts, or analogs: Tetrahydrocannabinol (THC), tetrahydrocannabinolic acid (THCA), Tetrahydrocannabivarin(THCV), Cannabichromene(CBC), cannabidiolic acid (CBDA), Cannabichromanon (CBCN), Cannabidiol (CBD), Cannabielsoin (CBE), Cannabidivarin (CBDV), Cannbifuran (CBF), Cannabigerol (CBG), Cannabicyclol (CBL), Cannabinol (CBN), Cannabinodiol (CBND), Cannabitriol (CBT), Cannabivarin (CBV), and Isocanabinoids.

“Cannabis” or “cannabis plant” refers to any species in the Cannabis genus that produces cannabinoids, such as Cannabis sativa and interspecific hybrids thereof.

The disclosure provides methods for crossing a first plant with a second plant. As used herein, the term “cross”, “crossing”, “cross pollination” or “cross-breeding” refer to the process by which the pollen of one flower on one plant is applied (artificially or naturally) to the ovule (stigma) of a flower on another plant. Backcrossing is a process in which a breeder repeatedly crosses hybrid progeny, for example a first generation hybrid (F1), back to one of the parents of the hybrid progeny. Backcrossing can be used to introduce one or more single locus conversions from one genetic background into another.

The disclosure provides plant cultivars. As used herein, the term “cultivar” means a group of similar plants that by structural features and performance (i.e., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain and race are often used interchangeably by plant breeders, agronomists and farmers.

As used herein “CRISPR RNA (crRNA)” refers to the crRNA transcribed from interval spacer sequences that correlate to the sequences on plasmid or phage (prospacer). The crRNA plays a vital role in matching and recognizing the target DNA.

The term “enhancement,” “enhance,” “enhances,” “enhancing” or “increase” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or an increase in the specified activity of at least about 5%, 10%, 25%, 35%, 40%, 50%, 60%, 75%, 80%, 90%, 95%, 97%, 98%, 99% or 100%.

As used herein, “genome editing” (GE) is a technique that introduces mutations in the form of insertions and/or deletions (indels) or base substitutions in targeted sequences, so causing DNA modification.

As used herein “guide RNA” (gRNA) is a chimeric molecule that consists of tracrRNA and crRNA, anteceded by an 18-20-nt spacer sequence complementary to target DNA before PAM.

“His-Asn-His” (HNH) domain is one of the two endonuclease domains of Cas9 that functions to cleave the complementary strand of CRISPR RNA (crRNA).

“Homology-directed repair” (HDR) isa repair pathway that executes the precise sequence or insertion, or gene replacement, by adding a donor DNA template with sequence homology at a predicted DSB site. In the presence of an oligonucleotide template, HDR induces the specific replacement of genes or allows foreign DNA knock-ins.

As used herein, the term “inbreeding” refers to the production of offspring via the mating between relatives. The plants resulting from the inbreeding process are referred to as “inbred plants” or “inbreds.”

“Indels” is a general term used for insertion or deletion mutations.

The term “inhibit,” “diminish,” “reduce” or “suppress” refers to a decrease in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or a decrease or reduction in the specified activity of at least about 5%, 10%, 25%, 35%, 40%, 50%, 60%, 75%, 80%, 90%, 95%, 97%, 98%, 99% or 100%. These terms are intended to be relative to a reference or control.

“Modifying genetic material of the plant of the genus cannabis” includes excising or inactivating one or more single genes that produce THC. In one embodiment, one or more genes are chosen from available literature, and isolated from the closest relative with published sequence data. The synthetic DNA molecule embodied herein can be inserted into an expression cassette. This expression cassette can be inserted into the target Cannabis genera plant genome using a binary vector Agrobacterium mediated system. Small-scale transgenesis can be accomplished at a local scale with syringe infiltration, and in the whole plant via vacuum infiltration.

As used herein, “modulate,” “modulates” or “modulation” refers to enhancement (e.g., an increase) or inhibition (e.g., diminished, reduced or suppressed) of the specified activity or expression of a gene, polynucleotides, oligonucleotides, proteins, polypeptides, peptides or combinations thereof.

“Non-homologous end-joining” (NHEJ): a pathway that repairs DSBs and creates indels or mismatches leading to gene knockout and loss-of-function mutants. NHEJ-mediated repair can be used to generate point mutations via gene replacement when the target sequences of CRISPR/Cas9 are located in introns.

The disclosure provides offspring. As used herein, the term “offspring” refers to any plant resulting as progeny from a vegetative or sexual reproduction from one or more parent plants or descendants thereof. For instance, an offspring, plant may be obtained by cloning or selling of a parent plant or by crossing two parent plants and include the F1 or F2 or still further generations. An F1 is a first-generation offspring produced from parents at least one of which is used for the first time as donor of a trait, while offspring of second generation (F2) or subsequent generations (F3, F4, etc.) are specimens produced from F1's, F2's etc. An F1 may thus be (and usually is) a hybrid resulting from a cross between two true breeding parents (true-breeding is homozygous for a trait), while an F2 be (and usually is) an offspring resulting from self-pollination of said F1 hybrids.

“Protospacer adjacent motif” (PAM) is a 3-nt sequence located immediately downstream of the single guide RNA (sgRNA) target site, which plays an essential role in binding and for Cas9-mediated DNA cleavage. The PAMs are the various extended conserved bases at the 5′ or 3′ end of the protospacer.

As used herein, the term “plant” means a multicellular eukaryote of the kingdom Plantae, whether naturally occurring, completely manmade, or some combination thereof.

As used herein, the term “plant cell” refers to any totipotent plant cell from a cannabis plant. Plant cells of the present disclosure include cells from a cannabis plant shoot, root, stem, seed, stipule, leaf, petal, inflorescence, bud, ovule, bract, trichome, petiole, internode. In some embodiments, the disclosed plant cell is from a cannabis trichome.

As used herein, the term “plant of genus cannabis” means a plant belonging to the genus “cannabis” within the accepted biological taxonomical system, including the species Cannabis sativa, Cannabis indica, and Cannabis ruderalis.

As used herein, the term “plant part” refers to any part of a plant including but not limited to the embryo, shoot, root, stem, seed, stipule, leaf, petal, flower, inflorescence, bud, ovule, bract, trichome, branch, petiole, internode, bark, pubescence, tiller, rhizome, frond, blade, ovule, pollen, stamen, and the like. The two main parts of plants grown in some sort of media, such as soil or vermiculite, are often referred to as the “above-ground” part, also often referred to as the “shoots”, and the “below-ground” part, also often referred to as the “roots”. Plant parts may also include certain extracts such as kief or hash, which includes cannabis trichomes or glands. In some embodiments, plant part should also be interpreted as referring to individual cells derived from the plant.

“RuvC-like domain” is one of the two endonuclease domains of Cas9 that functions to cleave the complementary strand of dsDNA.

“Trans-activating crRNA” (tracrRNA) is a small trans-encoded RNA that stabilizes the structure and then activates the Cas9 for cleavage of the target DNA.

“Trichome” encompasses herein different types of trichomes, both glandular trichomes and/or non-glandular trichomes. “Trichome cells” refers to the cells making up the trichome structure, such as the gland, or secretory cells, base cells and stalk, or stripe cells, extra-cellular cavity and cuticle cells. Trichomes can also consist of one single cell.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged. The disclosure provides methods for obtaining plant lines. As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

### BRIEF DESCRIPTION OF THE FIGURES

FIG. 1A is a linear illustration of tetrahydrocannabinolic acid (THCA) gene and the position of the guide (g) RNAs 1 (SEQ ID NO: 1) and gRNA 2 (SEQ ID NO: 2) for targeting by CRISPR. FIG. 1B. Nucleotide sequence of the gRNAs. gRNA1: 5′-GAAGAATAAGACTACAGTACA TGG-3′ (SEQ ID NO: 1). gRNA2: 5′-GAACTTTGGTACACTGCTACC TGG-3 (SEQ ID NO: 2). 5′-AATTATGGCCTTGCGGCTGA-3′ FP (SEQ ID NO: 3). 5′-ACCCCAAATACGTGCTTGTG-3′ RP (SEQ ID NO: 4).

FIGS. 2A-2C are a series of blots demonstrating the expression of Spcas9 and gRNA1 and gRNA 2 in Px333+gRNA1+2c1 and c2 clones. FIG. 2A: Expression of SpCas9 by Western blot analysis in eukaryotic cells, TC60. FIGS. 2B and 2C: Production of gRNAs 1 and 2 to be used for editing THCA by CRISPR.

FIG. 3 is a protein sequence alignment for cannabidiolic acid (CBDA) and THCA. The amino acid sequence of THCA (SEQ ID NO: 5) and CBDA (SEQ ID NO: 6) illustrates homology at the amino acids of these two enzymes.

FIG. 4 is a gel showing the results from experiments targeting the THCAS gene with the CRISPR/Cas9 system.

FIG. 5 is a schematic representation of a general method for gene editing in a plant. Plant CRISPR/Cas9 products can be used for Agrobacterium-mediated plant transformation or biolistic microparticle bombardment or protoplast transformation. In this schematic, the products are based on the type IIA CRISPR/Cas9 derived from Streptococcus pyogenes. The native Cas9 coding sequence is codon optimized for expression in monocots and dicots, respectively. The monocot Cas9 constructs contain a monocot U6 promoter for sgRNA expression, and the dicot Cas9 constructs contain a dicot U6 promoter. The plant selection markers include hygromycin B resistance gene, neomycin phosphotransferase gene, and the bar gene (phosphinothricin acetyl transferase).

FIGS. 6A-6O is a schematic representation showing a general method of CRISPR/Cas9 genetic transformation of genes from gene selection to plant analysis. (FIG. 6A) Selection of the target gene. (FIG. 6B) Designing the single-guide RNA (sgRNA) for the target gene. (FIG. 6C) Vector construction. (FIG. 6D) Genetic transformation via Agrobacterium/ribonucleoprotein (RNP) for the delivery of CRISPR/Cas9. (FIG. 6E) Tissue culture (callus induction). (FIG. 6F) Plant regeneration from CRISPR/Cas9-mutated tissues. (FIG. 6G) Generation of TO CRISPR/Cas9-mutated transgenic plants. (FIG. 6H) Screening of transgenic plants by PCR. (FIG. 6I) Detection of on- and off-target efficiency of CRISPR/Cas9-mutated plants by T7E1. (FIG. 6J) Detection of on- and off-target efficiency by Sanger sequencing. (FIG. 6K) Different methods to detect on- and off-target efficiency. (FIG. 6L) Self-pollination of T0 transgenic plants for generation of homozygous T1 plants. (FIG. 6M) CRISPR/Cas9-mutated TO seeds. (FIG. 6N) Generation of transgene-free T1 progeny. (FIG. 6O) Phenotypic analysis of T1 plants and other analysis. Abbreviations: Cas9, CRISPR-associated nuclease 9; CRISPR, clustered regularly interspaced short palindromic repeat; crRNA, CRISPR RNA; tracrRNA, trans-activating CRISPR RNA.

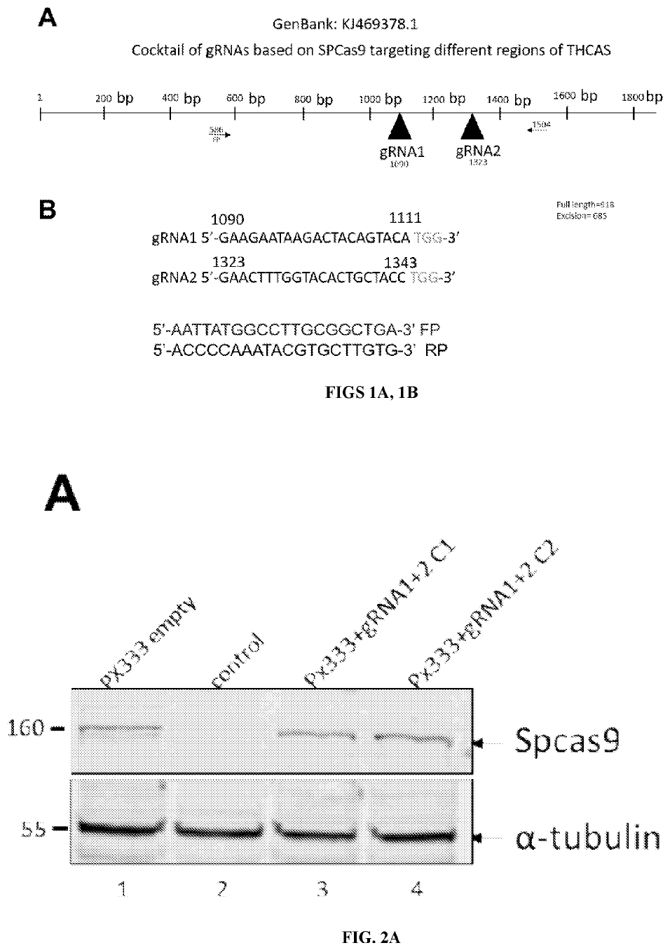

### DETAILED DESCRIPTION

Cannabis is a genus of flowering plant. Plants of genus cannabis include three different species: Cannabis sativa, Cannabis indica, and Cannabis ruderalis. Plants of genus cannabis have long been used for hemp fiber, for seed and seed oils, for medicinal purposes, and for psychoactive properties.

Cannabis is composed of at least 483 known chemical compounds, which include cannabinoids, terpenoids, flavonoids, nitrogenous compounds, amino acids, proteins, glycoproteins, enzymes, sugars and related compounds, hydrocarbons, simple alcohols, aldehydes, ketones, simple acids, fatty acids, simple esters, lactones, steroids, terpenes, non-cannabinoid phenols, vitamins, pigments, and elements. These compounds are secreted on the glandular trichomes. Cannabinoids are unique to the cannabis plant and there have been 100 cannabinoids that have been isolated as purified (single) molecules.

Most extraction processes aim to extract cannabinoids from the flowering parts of the cannabis plant, particularly tetrahydrocannabinol (THC). THC has many effects including relieving pain, treating glaucoma, relieving nausea, and as an antiemetic during treatments (see Regulation of Nausea and Vomiting by Cannabinoids, British Journal of Pharmacology; Parker, Rock, Linebeer). The latter is sold as the drug dronabinol, a pure isomer of THC, (−)-trans-A9-tetrahydrocannabinol which is manmade. The brand name in the US is Marinol.

Accordingly, there is a need to obtain CBD compositions which lack THC without the need to cultivate the cannabis plant. Furthermore, the purity of CBD can be affected by the presence of THC or even contaminates from the extraction process. The flowering parts of the cannabis plant include trichomes, which comprise the majority of the plant's secondary compounds, e.g., cannabinoids and terpenes. Trichomes can be separated from the plant by placing the whole plant in a fine mesh Screen Sifter and gently shaking so that the trichomes fall through the screen away from the plant. The crude trichomes are sometimes compressed into rounds known as hash or hashish.

Harvesting secondary compounds, e.g., cannabinoids and terpenes from a plant of the genus cannabis requires harvesting trichomes. Harvesting trichomes requires flowering a plant of the genus cannabis. From start to finish, harvesting secondary compounds from the trichomes of a plant of genus cannabis requires five stages of plant growth: Germination; Seeding; Vegetative Growth; Pre-Flowering; and Flowering.

To overcome these drawbacks, the invention embodied herein, is directed to the excision of the nucleotide sequences, which ultimately translates to the synthesis of THC, and develop Cannabis Sativa L phenotypes and strains devoid of the ability to produce THC. This phenotype development will significantly aid in the purity and safety of CBD product formulations, as well as protect American hemp harvests from testing THC positive, thereby eliminating any risk of THC contamination. Furthermore, with the utilization of CRISPR technology, other nucleotide sequences can be targeted within the chemotype composition of Cannabis Sativa L, which consists of over 500 active compounds potentially possessing medicinal qualities. Doing so will allow the ability to “program” plants, whereby phenotypes are developed in a “target specific” fashion aimed at specific treatment potentials. Ultimately, this will create an extremely valuable resource allowing for the development of natural, plant-based hemp products that are beneficial to mankind, can be produced more cost effectively, and greatly reduce, if not eliminate, potential deleterious effects.

In some embodiments, the present disclosure provides methods for obtaining plant genotypes comprising recombinant genes. As used herein, the term “genotype” refers to the genetic makeup of an individual cell, cell culture, tissue, organism (e.g., a plant), or group of organisms.

In some embodiments, the present disclosure provides homozygotes. As used herein, the term “homozygote” refers to an individual cell or plant having the same alleles at one or more loci.

In some embodiments, the present disclosure provides homozygous plants. As used herein, the term “homozygous” refers to the presence of identical alleles at one or more loci in homologous chromosomal segments.

In some embodiments, the present disclosure provides hemizygotes. As used herein, the term “hemizygotes” or “hemizygous” refers to a cell, tissue, organism or plant in which a gene is present only once in a genotype, as a gene in a haploid cell or organism, a sex-linked gene in the heterogametic sex, or a gene in a segment of chromosome in a diploid cell or organism where its partner segment has been deleted.

In some embodiments, the present disclosure provides heterozygotes. As used herein, the terms “heterozygote” and “heterozygous” refer to a diploid or polyploid individual cell or plant having different alleles (forms of a given gene) present at least at one locus. In some embodiments, the cell or organism is heterozygous for the gene of interest that is under control of the synthetic regulatory element.

The disclosure provides self-pollination populations. As used herein, the term “self-crossing”, “self-pollinated” or “self-pollination” means the pollen of one flower on one plant is applied (artificially or naturally) to the ovule (stigma) of the same or a different flower on the same plant.

The disclosure provides ovules and pollens of plants. As used herein when discussing plants, the term “ovule” refers to the female gametophyte, whereas the term “pollen” means the male gametophyte.

The disclosure provides methods for obtaining plants comprising recombinant genes through transformation. As used herein, the term “transformation” refers to the transfer of nucleic acid (i. e. a nucleotide polymer) into a cell. As used herein, the term “genetic transformation” refers to the transfer and incorporation of DNA, especially recombinant DNA, into a cell.

The present disclosure also relates to variants, mutants and modifications of the seeds, plant parts and/or whole plants of the cannabis plants of the present disclosure. Variants, mutants and trivial modifications of the seeds, plants, plant parts, plant cells of the present disclosure can be generated by methods well known and available to one skilled in the art, including but not limited to, mutagenesis (e.g., chemical mutagenesis, radiation mutagenesis, transposon mutagenesis, insertional mutagenesis, signature tagged mutagenesis, site-directed mutagenesis, and natural mutagenesis), knock-outs/knock-ins, antisense and RNA interference. For more information of mutagenesis in plants, such as agents, protocols, see Acquaah et al. (Principles of plant genetics and breeding, Wiley-Blackwell, 2007, ISBN 1405136464, 9781405136464, which is herein incorporated by reference in its entity).

The present disclosure also relates to a mutagenized population of the cannabis plants of the present disclosure, and methods of using such populations. In some embodiments, the mutagenized population can be used in screening for new cannabis lines that comprise one or more or all of the morphological, physiological, biological, and/or chemical characteristics of cannabis plants of the present disclosure. In some embodiments, the new cannabis plants obtained from the screening process comprise one or more or all of the morphological, physiological, biological, and/or chemical characteristics of cannabis plants of the present disclosure, and one or more additional or different new morphological, physiological, biological, and/or chemical characteristic.

The most common method for the introduction of new genetic material into a plant genome involves the use of living cells of the bacterial pathogen Agrobacterium tumefaciens to literally inject a piece of DNA, called transfer or T-DNA, into individual plant cells (usually following wounding of the tissue) where it is targeted to the plant nucleus for chromosomal integration. There are numerous patents governing Agrobacterium mediated transformation and particular DNA delivery plasmids designed specifically for use with Agrobacterium for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods Agrobacterium-mediated plant transformation that involve using vectors with no T-DNA are also well known to those skilled in the art and can have applicability in the present disclosure. See, for example, U.S. Pat. No. 7,250,554, which utilizes P-DNA instead of T-DNA in the transformation vector.

Direct plant transformation methods using DNA have also been reported. The first of these to be reported historically is electroporation, which utilizes an electrical current applied to a solution containing plant cells (M. E. Fromm et al., Nature 319, 791 (1986); H. Jones et al., Plant Mol. Biol., 13, 501 (1989) and H. Yang et al., Plant Cell Reports, 7, 421 (1988). Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. Nos. 5,204,253, 5,015,580). A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation and also for bacterial and animal transformation (There are other methods reported, and undoubtedly, additional methods will be developed. However, the efficiencies of each of these indirect or direct methods in introducing foreign DNA into plant cells are invariably extremely low, making it necessary to use some method for selection of only those cells that have been transformed, and further, allowing growth and regeneration into plants of only those cells that have been transformed.

For efficient plant transformation, a selection method must be employed such that whole plants are regenerated from a single transformed cell and every cell of the transformed plant canies the DNA of interest. These methods can employ positive selection, whereby a foreign gene is supplied to a plant cell that allows it to utilize a substrate present in the medium that it otherwise could not use, such as mannose or xylose (for example, refer U.S. Pat. Nos. 5,767,378; 5,994,629). More typically, however, negative selection is used because it is more efficient, utilizing selective agents such as herbicides or antibiotics that either kill or inhibit the growth of nontransformed plant cells and reducing the possibility of chimeras. Resistance genes that are effective against negative selective agents are provided on the introduced foreign DNA used for the plant transformation. For example, one of the most popular selective agents used is the antibiotic kanamycin, together with the resistance gene neomycin phosphotransferase (nptH), which confers resistance to kanamycin and related antibiotics (see, for example, Messing & Vierra, Gene 19: 259-268 (1982); Bevan et al., Nature 304: 184-187 (1983)). However, many different antibiotics and antibiotic resistance genes can be used for transformation purposes (refer U.S. Pat. Nos. 5,034,322, 6,174,724 and 6,255,560). In addition, several herbicides and herbicide resistance genes have been used for transformation purposes, including the bar gene, which confers resistance to the herbicide phosphinothricin (White et al., Nucl. Acids Res 18: 1062 (1990), Spencer at al., Theor Appl Genet 79: 625-6310990), U.S. Pat. Nos. 4,795,855, 5,378,824 and 6,107,549). In addition, the dhfr gene, which confers resistance to the anticancer agent methotrexate, has been used for selection (Bourouis et al., EMBO J. 2(7): 1099-1104 (1983).

Genes can be introduced in a site directed fashion using homologous recombination. Homologous recombination permits site specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513, 5,501,967 and 5,527,695.

In certain embodiments, cannabis plants are contacted with (i) gene editing agents which specifically excise the THC gene or inactivate the expression of the THC gene and (ii) gene editing complexes which are directed to gene regulatory regions of cannabinoids, e.g., cannabidiol (CBD) to enhance the expression of desired cannabinoids and/or (iii) expression vectors which express cannabinoid genes. This results not only in eliminating production of THC, but also produces plants which express higher amounts of cannabinoids as compared to a normal cannabis plant or normal control.

Plant Expresston Vectors: Vectors for delivery of the gene-editing agents for use in use in plants are known in the art. For a. review, see, for example, Hefferon K. (2017). Plant Virus Expression Vectors: A Powerhouse for Global Health. Biomedicines, 5(3), 44. doi.org/10.3390/biomedicines5030044, incorporated by reference herein in its entirety. Plant viruses have been engineered to express vaccines, monoclonal antibodies, and other therapeutic proteins. Plant virus expression vectors have been designed from the genomes of both positive-sense RNA viruses or single-stranded DNA viruses (Gleba Y. et al. (2007) Viral vectors for the expression of proteins in plants. Curr Opin Biotechnol. April; 18(2):134-41. Klimyuk V. et a. (2014) Production of recombinant antigens and antibodies in Nicotiana benthamiana using ‘magnifection’ technology: GMP-compliant facilities for small- and large-scale manufacturing. Curr Top Microbiol Immunol. 3750:127-54. Kagale S. at al. (2012) TMV-Gate vectors: gateway compatible tobacco mosaic virus based expression vectors for functional analysis of proteins. Sci Rep. 2012; 2( )874).

Second generation virus expression vectorshave no size limitation of foreign genes, have improved production levels, and overcome both host plant species and tissue restrictions. These ‘deconstructed vectors’ are composed solely of the foreign gene of interest and the minimum virus components that are required for replication (Gleba Y. et al. (2004) Engineering viral expression vectors for plants: the ‘full virus’ and the ‘deconstructed virus’ strategies. Curr Opin Plant Biol. 2004 April; 7(2):182-8. Gleba Y. et al. (2005) Magnifection—a new platform for expressing recombinant vaccines in plants. Vaccine. 2005 Mar. 18; 23(17-142042-8). As a consequence of the removal of genes essential to virus transport and assembly, for example, deconstructed vectors must be delivered to the host plant by alternative means, such as vacuum infiltration of the agrobacterium suspension that harbors the expression vector into plant leaves (Leuzinger K. et at., (2013) Efficient agroinfiltration of plants for high-level transient expression of recombinant proteins. J Vis Exp. July 23; 77)). This synchronous production of the desired pharmaceutical protein in all plant tissues can increase protein production in a reduced time period.

Examples of virus vectors include, without limitation, Tobamoviruses (e.g. Tobacco mosiaic virus), Comoviruses (e.g. Comovirus Cowpea mosaic virus), Potexviruses (e.g. Potato Virus X), Geminiviruses (e.g. Bean yellow dwarf virus) (Hefferon K. (2017). Plant Virus Expression Vectors: A Powerhouse for Global Health. Biomedicines, 5(3), 44). Examples of plasmid vectors include the pDGE Dicot Genome Editing Kit available from Addgene (Watertown, Mass.) (Ordon J. et al. (2016) Generation of chromosomal deletions in dicotyledonous plants employing a user-friendly genome editing toolkit. Plant J. 2016 Aug. 31. doi.: 10.1.1.11/tpj.13319. [Epub ahead of print] PubMed PMID: 27579989). Another example of a vector is the pCambia vector whereby the backbone is derived from the pPZP vectors (Abeam, Cambridge, UK). pCambia vectors are driven by a double-enhancer version of the CaMV35S promoter and terminated by the CaMV35S polyA signal. Reporter genes feature a hexa-Histidine tag at the C-terminus to enable simple purification on immobilized metal affinity chromatography resins. This vector contains a fully functional gusA reporter construct for simple and sensitive analysis of gene function or presence in regenerated plants by GUS assay. The construct uses E. coli gusA with an intron (from the castor bean catalase gene) inside the coding sequence to ensure that expression of glucuronidase activity is derived from eukaryotic cells, not from expression by residual A. tumefaciens cells (Tuhaise S. et al. (2019) “Establishment of a transformation protocol for Uganda's yellow passion fruit using the GUS gene.” African J Biotech 18(20), pp. 416-425).

Methods of Producing Transgenic Plants: Methods of producing transgenic plants are well known to those of ordinary skill in the art. Transgenic plants can now be produced by a variety of different transformation methods including, but not limited to, electroporation: microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,559,318; 5,641,664; 5,736,369 and 5,736,369; and International Patent Application Publication Nos. WO/2002/038779 and WO/2009/117555; (Lu et al., Plant Cell Reports, 2008, 27:273-278) Watson et al., Recombinant DNA, Scientific American Books (1992); Hinchee et al., Bio/Tech. 6:915-922 (1988); McCabe et al. Bio/Tech. 6:923-926 (1988); Toriyama et al., Bio/Tech. 6: 1072-1074 (1988); Fromm et al., Bio/Tech. 8:833-839 (1990); Mullins et al. Bio/Tech. 8:833-839 (1990); Hiei et al., Plant Molecular Biology 35:205- 218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety. Other references teaching the transformation of cannabis plants and the production of callus tissue include Rahaijo et al 2006, “Callus Induction and Phytochemical Characterization of Cannabis sativa Cell Suspension Cultures”, Indo. J. Chem 6 (1) 70-74; and “The biotechnology of Cannabis sativa” by Sam R. Zwenger, electronically published April, 2009.

Microprojectile bombardment is also known as particle acceleration, biolistic bombardment, and the gene gun (BIOLISTIC® Gene Gun). The gene gun is used to shoot pellets that are coated with genes (e.g., for desired traits) into plant seeds or plant tissues in order to get the plant cells to then express the new genes. The gene gun uses an actual explosive (.22 caliber blank) to propel the material. Compressed air or steam may also be used as the propellant. The BIOLISTIC® Gene Gun was invented in 1983-1984 at Cornell University by John Sanford, Edward Wolf, and Nelson Allen. It and its registered trademark are now owned by E. I. du Pont de Nemours and Company. Most species of plants hare been transformed using this method.

Agrobacterium tumefaciens is a naturally occurring bacterium that is capable of inserting its DNA (genetic information) into plants, resulting in a type of injury to the plant known as crown gall. Most species of plants can now be transformed using this method, including cucurbitaceous species. A transgenic plant formed using Agrobacterium transformation methods typically contains a single gene on one chromosome, although multiple copies are possible. Such transgenic plants can be referred to as being hemizygous for the added gene. A more accurate name for such a plant is an independent segregant, because each transformed plant represents a unique T-DNA integration event (U.S. Pat. No. 6,156,953). A transgene locus is generally characterized by the presence and/or absence of the transgene e.g. THC. A heterozygous genotype in which one allele corresponds to the absence of the transgene is also designated hemizygous (U.S. Pat. No. 6,008,437).

General transformation methods, and specific methods for transforming certain plant species (e.g., maize) are described in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830, each of which is incorporated herein by reference in its entirety for all purposes.

Non-limiting examples of methods for transforming cannabis plants and cannabis tissue culture methods are described in Zweger (The Biotechnology of Cannabis sativa, April 2009); MacKinnon (Genetic transformation of Cannabis sativa Linn: a multipurpose fiber crop. doctoral thesis, University of Dundee, Scotland, 2003). MacKinnon at al. (Progress towards transformation of fiber hemp, Scottish Crop Research, 2000), and US 20120311744, each of which is herein incorporated by reference in its entirety for all purposes. The transformation can be physical, chemical and/or biological.

In some embodiments, the present disclosure teaches the genetic modification of Specialty Cannabis. In some embodiments, the Specialty Cannabis of the present disclosure comprise one or more transgenes, or DNA edits. Thus in some embodiments, the present disclosure teaches transformation of plants (e.g., via agrobacterium, gene gun, or other delivery mechanism). In other embodiments, the present disclosure teaches gene editing with CRISPR.

Gene Editing Agents: Compositions of the disclosure include at least one gene editing agent, comprising CRISPR-associated nucleases such as Cas9 and Cpf1 gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof. See Schiffer, 2012, J Virol 88(17):8920-8936, incorporated by reference.

The composition can also include C2c2-the first naturally-occurring CRISPR system that targets only RNA. The Class 2 type VI-A CRISPR-Cas effector “C2c2” demonstrates an RNA-guided RNase function. C2c2 from the bacterium Leptotrichia shahii provides interference against RNA phage. In vitro biochemical analysis show that C2c2 is guided by a single crRNA and can be programmed to cleave ssRNA targets carrying complementary protospacers. In bacteria, C2c2 can be programmed to knock down specific mRNAs. Cleavage is mediated by catalytic residues in the two conserved HEPN domains, mutations in which generate catalytically inactive RNA-binding proteins. These results demonstrate the capability of C2c2 as a new RNA-targeting tools.

C2c2 can be programmed to cleave particular RNA sequences in bacterial cells. The RNA-focused action of C2c2 complements the CRISPR-Cas9 system, which targets DNA, the genomic blueprint for cellular identity and function. The ability to target only RNA, which helps carry out the genomic instructions, offers the ability to specifically manipulate RNA in a high-throughput manner-and manipulate gene function more broadly.

CRISPR/Cpf1 is a DNA-editing technology analogous to the CRISPR/Cas9 system, characterized in 2015 by Feng Zhang's group from the Broad Institute and MIT. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations.

As referenced above, Argonaute is another potential gene editing system. Argonautes are a family of endonucleases that use 5′ phosphorylated short single-stranded nucleic acids as guides to cleave targets (Swarts, D. C. et al. The evolutionary journey of Argonaute proteins. Nat. Struct. Mol. Biol. 21, 743-753 (2014)). Similar to Cas9, Argonautes have key roles in gene expression repression and defense against foreign nucleic acids (Swarts, D. C. et al. Nat. Struct. Mol. Biol. 21, 743-753 (2014); Makarova, K S., et al. Biol. Direct 4, 29 (2009). Molloy, S. Nat. Rev. Microbiol. 11, 743 (2013); Vogel, J. Science 344, 972-973 (2014). Swarts, D. C. et al. Nature 507, 258-261 (2014); Olovnikov, I., et al. Mol. Cell 51, 594-605 (2013)). However, Argonautes differ from Cas9 in many ways Swarts, D. C. et al. The evolutionary journey of Argonaute proteins. Nat. Struct. Mol. Biol. 21, 743-753 (2014)). Cas9 only exist in prokaryotes, whereas Argonautes are preserved through evolution and exist in virtually all organisms; although most Argonautes associate with single-stranded (ss)RNAs and have a central role in RNA silencing, some Argonautes bind ssDNAs and cleave target DNAs (Swarts, D. C. et al. Nature 507, 258-261 (2014); Swarts, D. C. et al. Nucleic Acids Res. 43, 5120-5129 (2015)). guide RNAs must have a 3′ RNA-RNA hybridization structure for correct Cas9 binding, whereas no specific consensus secondary structure of guides is required for Argonaute binding; whereas Cas9 can only cleave a target upstream of a PAM, there is no specific sequence on targets required for Argonaute. Once Argonaute and guides bind, they affect the physicochemical characteristics of each other and work as a whole with kinetic properties more typical of nucleic-acid-binding proteins (Salomon, W. E., et al. Cell 162, 84-95 (2015)).

CRISPR Associated Endonucleases: CRISPR (Clustered Regularly Interspaced Short Palindromic Repeats) is found in bacteria and is believed to protect the bacteria from phage infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al., Annu Rev Genet (2011) 45:273-297; Wiedenheft et al., Nature (2012) 482:331-338); Jinek M et al., Science (2012) 337:816-821; Cong L et al., Science (2013) 339:819-823; Jinek M et al., (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

In general, the CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains. The mechanism through which CRISPR/Cas9-induced mutations inactivate the THC can vary. For example, the mutation can affect THC gene expression or excises the gene in-whole or in part. The mutation can comprise one or more deletions. The size of the deletion can vary from a single nucleotide base pair to about 10,000 base pairs. In some embodiments, the deletion can include all or substantially all of the THC sequence. The mutation can also comprise one or more insertions, that is, the addition of one or more nucleotide base pairs to the THC sequence. The size of the inserted sequence also may vary, for example from about one base pair to about 300 nucleotide base pairs. The mutation can comprise one or more point mutations, that is, the replacement of a single nucleotide with another nucleotide. Useful point mutations are those that have functional consequences, for example, mutations that result in the conversion of an amino acid codon into a termination codon, or that result in the production of a nonfunctional protein.

In embodiments. the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

Three types (I-III) of CRISPR systems have been identified. CRISPR clusters contain spacers, the sequences complementary to antecedent mobile elements. CRISPR clusters are transcribed and processed into mature CRISPR RNA (crRNA). In embodiments, the CRISPR/Cas system can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

A variety of CRISPR systems have been generated for efficient gene editing. The Cas9 variant CjCas9, derived from Campylobacter jejuni, is composed of 984 amino acid residues (2.95 kbp) and has been used for efficient gene editing in vitro and in vivo. CjCas9 is highly specific and cuts only a limited number of sites in the genomes of mouse or human. Delivered through adeno-associated virus (AAV), it has been shown to induce targeted mutations at high frequencies in retinal pigment epithelium (RPE) cells or mouse muscle cells.

Cas13 is a recently identified CRISPR effector and CRISPR/Cas13 can target specific viral RNAs and endogenous RNAs in plants cells (Wolter, F. and Puchta, H. (2018) The CRISPR/Cas revolution reaches the RNA world: Cas13, a new Swiss Army knife for plant biologists. Plant J. 94, 767-775). The Cas13 system has high RNA target specificity and efficiency (Abudayyeh, O. O. et al. (2017) RNA targeting with CRISPR-Cas13. Nature 550, 280-284). CRISPR/Cas13a has been considered as an entirely new CRISPR type that belongs to class II type VI.

Accordingly, in certain embodiments. The RNA endonuclease-guided endonuclease is CRISPR/Cas13. Due to the presence of higher eukaryotes and prokaryotes nucleotide-binding (HEPN) domains, it is associated with RNase activity. The CRISPR/Cas9 and CRISPR/LshCas13a systems have each been used to create resistance against potyvirus (an RNA virus) in plants, which indicates that this system can be used in agricultural and biotechnological applications (Aman, R. et al. (2018) RNA virus interference via CRISPR/Cas13a system in plants. Genome Biol. 19, 1).

Phage-assisted continuous evolution was used to develop an SpCas9 variant, xCas9(3.7), which recognizes a broader range of protospacer adjacent motifs (PAMs) (Rees, H. A. and Liu, D. R. (2018) Base editing: precision chemistry on the genome and transcriptome of living cells. Nat. Rev. Genet. 19, 770-788). xCas9 possesses a higher DNA specificity and editing efficiency, lower off-target activity, and broader PAM compatibility (including NG, GAA, and GAT) than does SpCas9, from which it is derived (Hu, J. H. et al. (2018) Evolved Cas9 variants with broad PAM compatibility and high DNA specificity. Nature 556, 57-63).

In one embodiment, the RNA-guided endonuclease is derived from a type II CRISPR/Cas system. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

The CRISPR-associated endonuclease Cas9 nuclease can have a nucleotide sequence identical to the wild type Streptococcus pyogenes sequence. The CRISPR-associated endonuclease may be a sequence from other species, for example other Streptococcus species, such as thermophiles. The Cas9 nuclease sequence can be derived from other species including, but not limited to: Nocardiopsis dassonvillei, Streptomyces pristinaespiralis, Streptomyces viridochromogenes, Streptomyces roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficle, Finegoldia magna, Natranaerobius thermophiles, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Pseudomonas aeruginosa, Escherichia coli, or other sequenced bacteria genomes and archaea, or other prokaryotic microorganisms may also be a source of the Cas9 sequence utilized in the embodiments disclosed herein.

The wild type Streptococcus pyogenes Cas9 sequence can be modified. The nucleic acid sequence can be codon optimized for efficient expression in plant cells Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765 or Cas9 amino acid sequence of PX330 or PX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

The Cas9 nuclease sequence can be a mutated sequence. For example, the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks.

The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In addition to the wild type and variant Cas9 endonucleases described, embodiments of the disclosure also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI:10.1126/science.aad5227).

Three variants found to have the best cleavage efficiency and fewest off-target effects: SpCas9(K855A), SpCas9(K810A/K1003A/R1060A) (a.k.a. eSpCas9 1.0), and SpCas9(K848A/K1003A/R1060A) (a.k.a. eSPCas9 1.1) are employed in the compositions. The invention is by no means limited to these variants, and also encompasses all Cas9 variants (Slaymaker, I. M. et al. (2015)).

The present disclosure also includes another type of enhanced specificity Cas9 variant, “high fidelity” spCas9 variants (HF-Cas9) (Kleinstiver, B. P. et al., 2016, Nature. DOI: 10.1038/nature16526).

As used herein, the term “Cas” is meant to include all Cas molecules comprising variants, mutants, orthologues, high-fidelity variants and the like.

CRISPR/Cas vectors for use in plants can also be obtained commercially, (Millipore Sigma) which can be used in Agrobacterium-mediated plant transformation or biolistic microparticle bombardment or protoplast transformation.

CRISPR/Cas9 technology has been used to modify a wide range of plant species (Hakim Manghwar et al., Trends in Plant Science, December 2019, Vol. 24, No. 12 doi.org/10.1016/j.tplants.2019.09.006, incorporated herein by reference in its entirety), including Arabidopsis, rice, wheat (Triticum aestivum), maize, soybean (Glycine max), sorghum, cotton (Gossypium hirsutum L.), rapeseed (Brassica napus L., barley (Hordeum vulgare L.), Nicotiana benthamiana, tomato (Solanum lycopersicum L.), potato (Solanum tuberosum), sweet orange (Citrus sinensis L.), cucumber (Cucumis sativus L.), wild cabbage (Brassica oleracea L.), wild legume (Lotus japonicus L.), lettuce (Lactuca sativa L.), Medicago truncatula, Marchantia polymorpha, tobacco (Nicotiana tabacum L.), Nicotiana attenuata, Petunia hybrida, grape (Vitis vinifera L.), apple (Malus pumila), tropical staple cassava (Manihot esculenta), watermelon (Citrullus lanatus). There have been multiple examples of the application of CRISPR/Cas9 editing, as follows.

Targeted Mutagenesis: As described above, the CRISPR/Cas system can induce sequence-specific mutagenesis to interrupt genes to evaluate their functions and be used for trait improvement in crops (Scheben, A. et al. (2017) Towards CRISPR/Cas crops—bringing together genomics and genome editing. New Phytol. 216, 682-698). By mutation of its nuclease domains, Cas9 can be transformed into a DNA-binding protein. The consequence is that its DNA binding activity remains intact, whereas the DNA cleavage activity is deactivated. Direct or indirect fusion of this ‘dead’ Cas9 (dCas9) nuclease to an effector domain can be utilized to guide fusion proteins to specific sites in the genome (Konermann, S. et al. (2015) Genome-scale transcriptional activation by an engineered CRISPR-Cas9 complex. Nature 517, 583-588). This allows the exploitation of CRISPR/Cas for various site-specific modifications, including epigenetic changes (Hilton, I. B. et al. (2015) Epigenome editing by a CRISPR-Cas9-based acetyltransferase activates genes from promoters and enhancers. Nat. Biotechnol. 33, 510-517), regulation of gene expression (Tang, X. et al. (2017) A CRISPR-Cpf1 system for efficient genome editing and transcriptional repression in plants. Nat. Plants 3, 17018), and base editing (BE) without induction of DSB, such as facilitated by fusion with deaminases in rice, wheat, and maize (Zong, Y. et al. (2017) Precise base editing in rice, wheat and maize with a Cas9-cytidine deaminase fusion. Nat. Biotechnol. 35, 438-440) or imaging of genomic loci in live leaf cells of N. benthamiana (Dreissig, S. et al. (2017) Live-cell CRISPR imaging in plants reveals dynamic telomere movements. Plant J. 91, 565-57380).

Multiplex Gene-Editing: CRISPR has the potential to create mutations simultaneously at more than one genomic site by using multiple sgRNAs, in any organism. CRISPR/Cas9 has also been used for multiplex gene editing, which enables the rapid stacking of multiple traits in an elite variety background (Yin, K. et al. (2017) Progress and prospects in plant genome editing. Nat. Plants 3, 17107). Multiplex gene editing also provides a powerful tool for targeting multiple members of multigene families. It can be achieved in two ways, by either constructing multiple gRNA expression cassettes in separate vectors or assembling various sgRNAs in a single vector (Wang, C. et al. (2019) Clonal seeds from hybrid rice by simultaneous genome engineering of meiosis and fertilization genes. Nat Biotechnol. 37, 283-28).

Gene Regulation—CRISPR Interference and Activation: The CRISPR interfering (CRISPRi) system is used as an orthogonal system in a variety of living organisms; the requirements are only a coexpression of a catalytically inactive Cas9 protein and a modified sgRNA, designed with a complementary region to any gene of interest. The CRISPRi system is derived from the S. pyogenes CRISPR pathway. The complex comprising Cas9 and sgRNA binds to DNA elements complementary to the sgRNA and causes a steric block that stops transcript elongation by RNA polymerase, so repressing the target gene. Therefore, CRISPRi has been considered as an effective and precise genome-targeting platform for transcription control without changing the target DNA sequence (Larson, M. H. et al. (2013) CRISPR interference (CRISPRi) for sequence-specific control of gene expression. Nat. Protoc. 8, 2180-2196). dCas9 is a useful and robust tool for the regulation of transcription levels of any target gene. The gRNA directs the binding of dCas9 to any genomic locus that can efficiently stop the progress of RNA polymerase to the downstream gene.

In various plant species, an efficient multiplex transcriptional activation has been successfully developed using the CRISPRAct2.0 and mTALE-Act systems. These tools can activate more than four genes at the same time and can be used to evaluate positive feedback transcriptional loops and the control of tissue-specific gene activation (Lowder, L. G. et al. (2018) Robust transcriptional activation in plants using multiplexed CRISPR-Act2. 0 and mTALE-act systems. Mol. Plant 11, 245-256); however, it does introduce more off-target effects. To solve this problem, a potent transcriptional activation tool termed dCas9-TV has been developed using VP128 (which possesses an additional VP64 moiety, which is an activation domain) that was joined to six copies each of plant-specific activation domains (ethylene response factor 2m and EDLL) and guided by a single sgRNA. This assembly promoted up to 55-fold activation of the target gene compared with the conventional dCas9-VP64 system (Li, Z. et al. (2017) A potent Cas9-derived gene activator for plant and mammalian cells. Nat. Plants 3, 930-936).

Epigenetic Modifications: Epigenetic and post-translational protein modifications, for example, DNA and histone acetylation/methylation, ubiquitination, SUMOylation, and phosphorylation, can alter chromatin structure and regulate gene expression patterns (Yamamuro, C. et al. (2016) Epigenetic modifications and plant hormone action. Mol. Plant 9, 57-70). The dCas9 fusion proteins can be used as sequence-specific synthetic epigenome converters, which alter local epigenetic status and the expression of related genes. dCas9 fused to epigenetic regulatory factors involved in histone acetylation, or methylation of DNA, can be used to modulate chromatin activity and gene expression patterns involved in plant development and environmental adaptation (Shrestha, A. et al. (2018) Cis-trans engineering advances and perspectives on customized transcriptional regulation in plants. Mol. Plant 11, 886-898). Recently, targeted DNA methylation or demethylation has been achieved in Arabidopsis (Gallego-Bartolomé, J. et al. (2018) Targeted DNA demethylation of the Arabidopsis genome using the human TET1 catalytic domain. Proc. Natl. Acad. Sci. U. S. A. 115, E2125-E2134). The histone demethylase Lys-specific histone demethylase 1 (LSD1) fused to Neisseria meningitidis dCas9 has been used for experimentally controlling gene repression (Dominguez, A. A. et al. (2016) Beyond editing: repurposing CRISPR—Cas9 for precision genome regulation and interrogation. Nat. Rev. Mol. Cell Biol. 17, 5-15).

Gene Replacement and Gene Knock-in: Double stranded breaks (DSBs) at targeted genome sites are repaired either by dependency on homology-directed repair (HDR) (also known as targeted integration (Wilson, L. O. et al. (2018) The current state and future of CRISPR-Cas9 gRNA design tools. Front. Pharmacol. 9, 749) or nonhomologous end-joining (NHEJ, which can allow gene replacement or gene knockout, respectively (Yin, K. et al. (2017) Progress and prospects in plant genome editing. Nat. Plants 3, 17107). CRISPR/Cas has successfully been used for gene replacement in plants (Schaeffer, S. M. and Nakata, P. A. (2015) CRISPR/ Cas9-mediated genome editing and gene replacement in plants: transitioning from lab to field. Plant Sci. 240, 130-142). One example is the replacement of the endogenous 5-enolpyruvylshikimate-3-phosphate synthase (OsEPSPS) in rice with a gene encoding a form of the protein tolerant to the herbicide glyphosate. HDR-mediated gene replacement has also been achieved in N. benthamiana protoplasts

Guide Nucleic Acid Sequences: Guide RNA sequences according to the present disclosure can be sense or anti-sense sequences. The specific sequence of the gRNA may vary, but, regardless of the sequence, useful guide RNA sequences will be those that minimize off-target effects while achieving high efficiency and complete ablation of the THC gene. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologues may have different PAM specificities. For example, Cas9 from S. thermophilus requires 5′-NNAGAA for CRISPR 1 and 5′-NGGNG for CRISPR3 and Neiseria meningitidis requires 5′-NNNNGATT. The specific sequence of the guide RNA may vary, but, regardless of the sequence, useful guide RNA sequences will be those that minimize off-target effects while achieving high efficiency and complete ablation of the THC gene. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs. In certain embodiments, the composition comprises multiple different gRNA molecules, each targeted to a different target sequence. In certain embodiments, this multiplexed strategy provides for increased efficacy. These multiplex gRNAs can be expressed separately in different vectors or expressed in one single vector.

The gene to be excised can be any desired gene. In certain embodiments, an exogenous gene is incorporated so that the plant produces a desired product. In certain embodiments, the amount of a certain gene product can be increased, for example CBD. In certain embodiments, the disclosure provides for a method of producing secondary compounds in a plant of genus cannabis, comprising inducing trichome development in a plant of genus cannabis. In some embodiments, the secondary compounds are chosen from cannabinoids or terpenes.

### Methodology for the Screening of CRISPR/Cas System-Induced Mutants

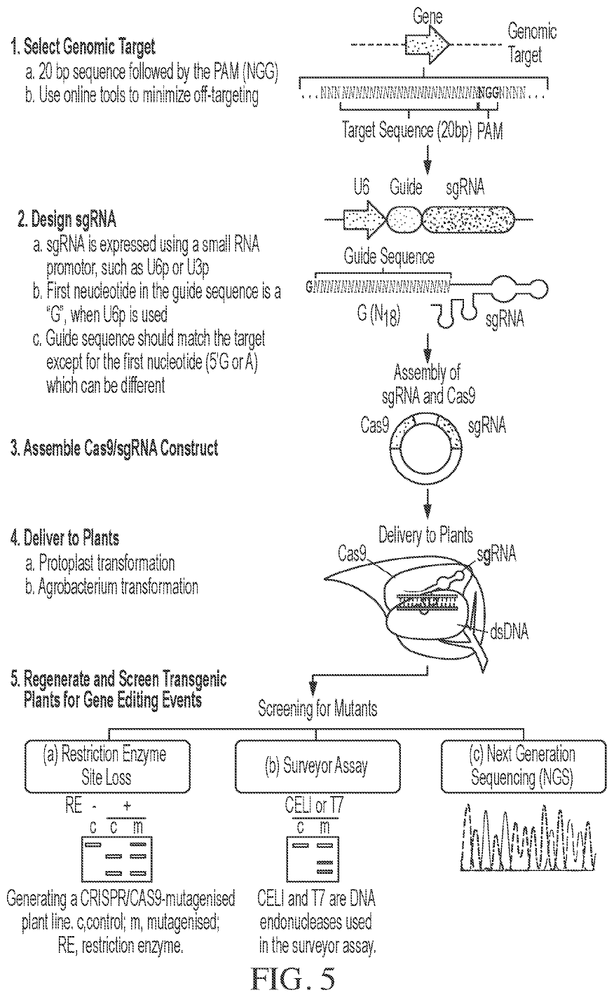

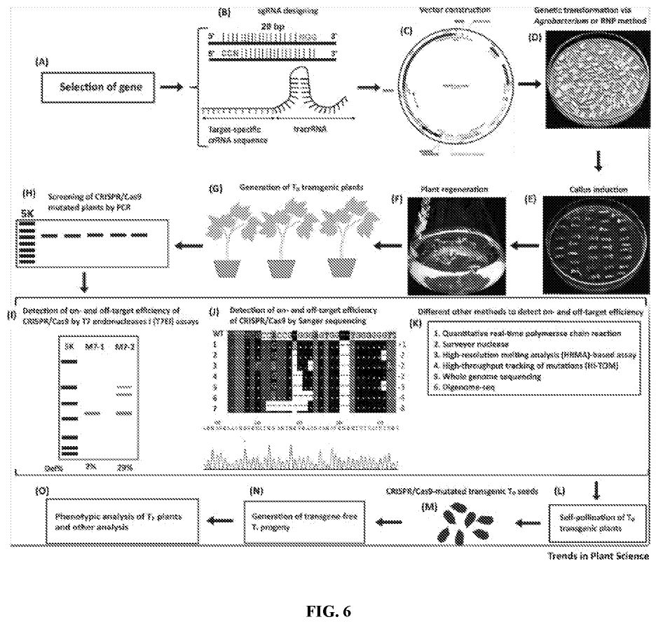

The first 20 nt of chimeric sgRNA and the PAM determine the target specificity of the CRISPR/Cas9 system. Efficient screening methods are crucial for the identification of induced mutations to analyze various genome-edited regenerated plants. A general protocol starting from selecting the target gene to genetic transformation by CRISPR/Cas9 system is illustrated in FIGS. 5 and 6.

qPCR: Mutated DNA sequences may be easily determined by amplifying the locus and sequencing the PCR products. qPCR can be used to distinguish homozygous and heterozygous mutations, and this approach has been validated in several plant species, including Arabidopsis (Arabidopsis thaliana), maize (Zea mays), sorghum (Sorghum bicolor), and rice (Oryza sativa) (Peng, C. et al. (2018) High-throughput detection and screening of plants modified by gene editing using quantitative real-time PCR. Plant J. 95, 557-567).

Surveyor Nuclease and T7 Endonuclease I (T7EI) Assays: SURVEYOR™ nuclease (Transgenomic Inc., Omaha, Neb., USA) belongs to the CEL family of mismatch-specific nucleases obtained from celery (Apium graveolens). It identifies and cleaves mismatches because of the occurrence of small indels or SNPs and cleaves both DNA strands downstream of the mismatch and detects indels of up to 12 nt (Qiu, P. et al. (2004) Mutation detection using SURVEYOR™ nuclease. Biotechniques 36, 702-707). The Surveyor nuclease and T7EI assays are extensively used and considered appropriate for any target sequence. They recognize and digest mismatched heteroduplex DNA. T7E1 can recognize and cleave various dsDNA molecules if their structure is curved and able to bend further (Déclais, A. C. et al. (2006) Structural recognition between a four-way DNA junction and a resolving enzyme. J. Mol. Biol. 359, 1261-1276).

High Resolution Melting Analysis (HRMA)-Based Assay: The HRMA assay involves DNA sequence amplification by qPCR covering about 90-200 bp of the genomic target, incorporating fluorescent dye followed by amplicon melt curve analysis (Wang, K. et al. (2015) Research of methods to detect genomic mutations induced by CRISPR/Cas systems. J. Biotechnol. 214, 128-132). HRMA is considered the most sensitive and simple method and compatible with a high-throughput screening format (96-well microliter plates). The whole procedure for genomic DNA preparation and mutation detection takes less than 2 hours, because of the nondestructive nature of the method. Further sequencing and gel electrophoresis can be used to analyze amplicons (Zischewski, J. et al. (2017) Detection of on-target and off-target mutations generated by CRISPR/Cas9 and other sequence-specific nucleases. Biotechnol. Adv. 35, 95-104).

High-Throughput Tracking of Mutations (Hi-TOM): Hi-TOM is an online tool (hi-tom.net/hi-tom/) that is used for the precise and quantitative detection of mutations caused by the CRISPR system. Hi-TOM does not require any additional data analysis or complex parameter configuration. It is easy to use and requires no specialist expertise in bioinformatics or next-generation sequencing (NGS). It has been found to be a more reliable and sensitive tool through analysis of human cells and rice tissues. Because of its convenience and simplicity, this tool has become the most suitable high-throughput detection methodology for mutations induced by CRISPR/Cas systems (Liu, Q. et al. (2018) Hi-TOM: a platform for high-throughput tracking of mutations induced by CRISPR/Cas systems. Sci. China Life Sci. 62, 1-7).

Whole-Genome Sequencing (WGS) to Detect On- and Off-Targets: WGS is a most effective technique for the identification of various kinds of mutations, such as small indels, SNPs, and structural variations, including major deletions, inversions, duplications, and rearrangements (Veres, A. et al. (2014) Low incidence of off-target mutations in individual CRISPR-Cas9 and TALEN targeted human stem cell clones detected by whole-genome sequencing. Cell Stem Cell 15,27-30), and has already been exploited for detecting off-target mutations caused by Cas9 in various crops.

### EXAMPLES

### Example 1: Materials and Methods

### Designing and Cloning Cocktails of gRNAs Targeting THCAS Gene

The genomic sequence of THCAS gene (gene bank: KJ469378.1) was obtained from NCBI data base and cocktail of gRNAs based on SPcas9 targeting two different regions of THCAS was designed using Benching CRISPER design tool (benchling.com). The best gRNA candidates were selected based on the highest on target and the lowest off target cleavage scores. A pair of oligonucleotides for each targeting site were designed in forward and reverse orientation as follows: THCAS gRNA1Fw 5′-GAA GAA TAA GAC TAC AGT ACA-3′ and THCAS gRNA1Rev 5′-TGT ACT GTA GTC TTA TTC TTC-3′ for THCAS gRNA2 Fw 5′-GAA CTT TGG TAC ACT GCT ACC-3′ and THCAS gRNA2 Rev 5′-GGT AGC AGT GTA CCA AAG TTC-3′ Each oligonucleotide contains sticky ends for cloning in a tandem of sgRNAs U6 cassettes in pX333 plasmid (Plasmid #64073 by Addgene) after sequential cutting by BbsI and BsaI and the same plasmid expresses also the CRISPR endonuclease SpCas9. The ligation mixture was transformed into competent cells and the cloning of gRNAs were confirmed by Sanger sequencing.

### Expression of gRNAs

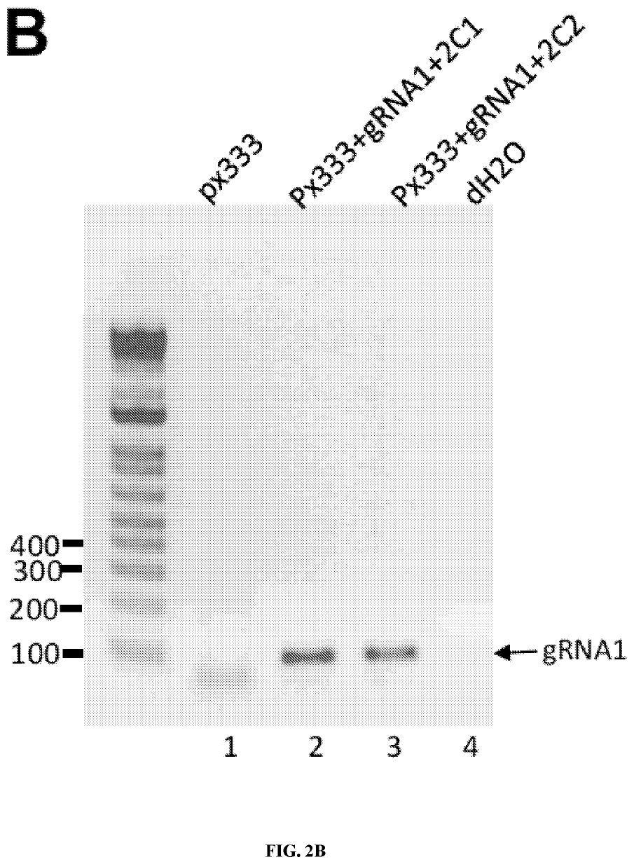

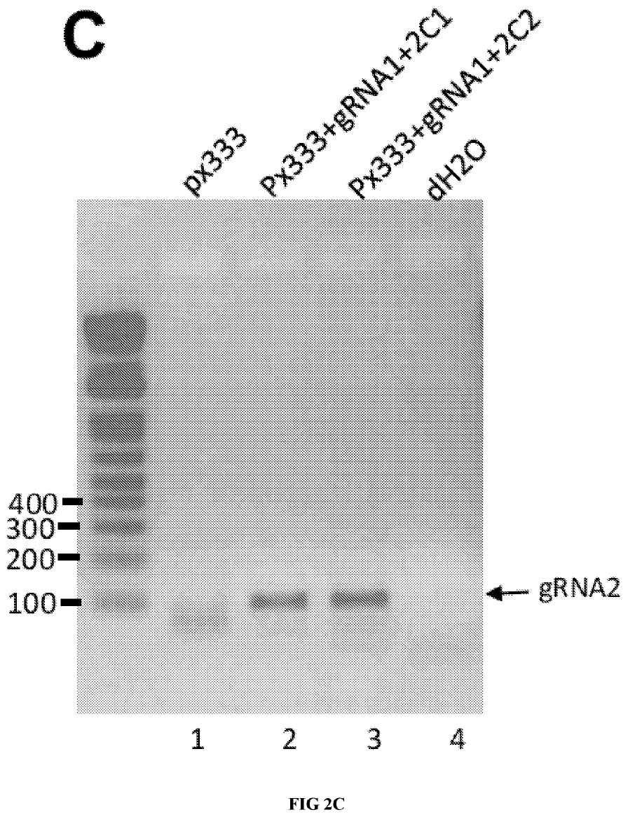

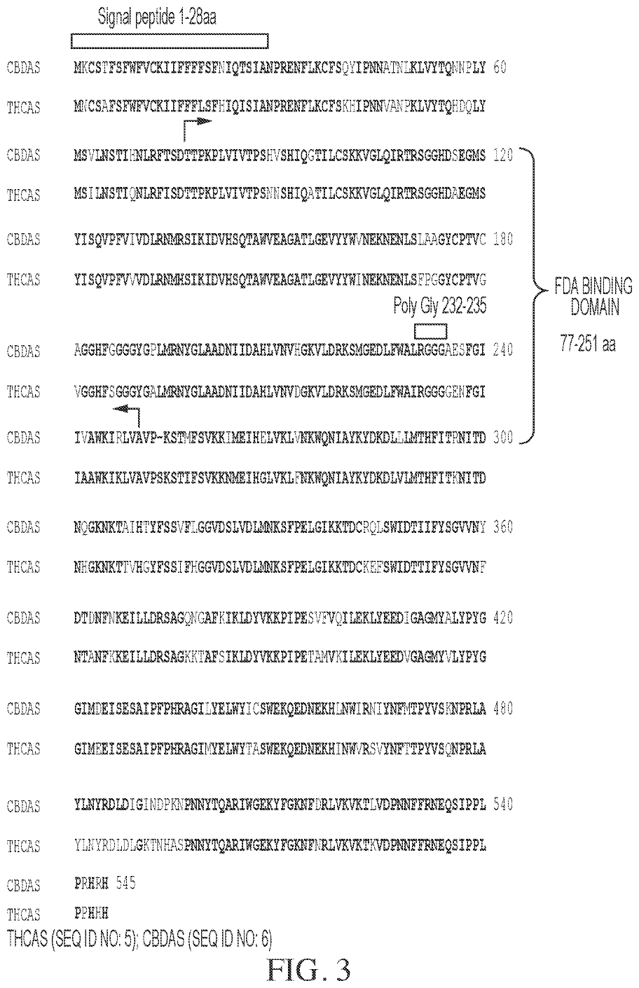

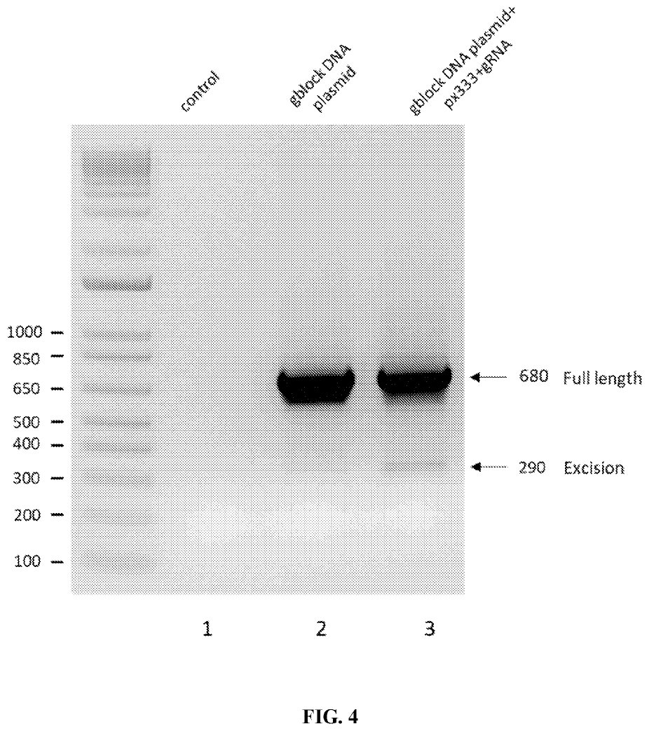

To determine the expression of gRNAs , total RNA was extracted from the cells using RNeasy Kit (Qiagen) 0.6 μg of RNA was used for M-MLV reverse transcription reaction (Invitrogen) using px333 based reverse primer (px 333-crRNA-3′) to generate cDNA. cDNA was subjected to PCR using fail Safe PCR kit and buffer D(Epicentre) under the following PCR conditions: 95° C. for 5 minutes, 30 cycles (95° C. 30 s, 55° C. 30 s, 72 ° C. 30 s, 72 ° C. 7 minutes). The PCR products were resolved in 1% agarose gel (FIGS. 2A-2C).

### Expression of SpCas9

Western-blot. Whole cell extract were prepared by incubation of the cells in TNN buffer (50 mM Tris pH 7.4, 150 mM NaCl, 1% Nonidet P-40, 5 mM EDTA pH 8, 1× protease inhibitor cocktail for mammalian cells (sigma) for 30 min at 4° C. by rotation and pre-cleared by centrifugation at maximum speed for 10 min at 4° C. 50 mg of lysate were denatured in 1× Laemli buffer and separated by SDS-polyacrylamide gel electrophoresis in tris-glycine buffer and transferred onto nitrocellulose membrane (BioRad). The membrane was blocked in 5% milk in PBST for 30 min and then incubated with the corresponding primary antibodies Flag tag mouse (1:1000). After washing with PBST, the membranes were incubated with conjugated goat anti-mouse antibody (1:5000) for 1 hours at room temperature. After washing the membrane 3 times for 5 min, the membrane was scanned and analyzed using an odyssey infrared system (LI-COR Bioscience)

### Validating the excision of THCAS gene by CRISPR/Cas9.

To verify the efficacy of the CRISPR/Cas9 targeting THCAS gene, we order gblock contains the entire THCAS coding sequence cloned plasmid Pucdt (integrated DNA Technologies) TC620 cells were cultured in DMEM medium containing 10% FBS and gentamycin (10 ug/ml). One day before transfection, the cells were plated in 6 well plate at the density of 0.3×10⁶. The next day, the cells were transfected with 2 ug control px333 empty plasmid or px333 containing THCAS gRNAs using fugene transfection reagent. 8 hours later media was removed and replaced with a fresh media .48 hours after the transfection, the cells were harvested and genomic DNA was isolated from the cells using Nucleospin Tissue kit (Macherey-Nagel) according to the protocol of the manufacturer. 300 ng of extracted DNA was subjected to PCR using fail Safe PCR kit and buffer D(Epicentre) under the following PCR conditions: 95° C. 5 minutes, 30 cycles (95° C. 30 s, 57 ° C. 30 s, 72 ° C. 30 s, 72 ° C. 7 minutes. The PCR products were resolved in 1% agarose gel and gel purified using QIAquick gel Extraction kit(QIAGEN) and cloned into TA vector (Invitrogen) and send for Sanger sequencing (Genewiz). The sequence alignment for full length and the Excision sequence was done using multiple sequence alignment program (ClustalW2).

### Example 2: Protoplast Development and Induction of Construct

Throughout the United States Industrial Hemp Industry cultivators and processors struggle to maintain Tetrahydrocannabinol (THC) threshold levels set forth by the Drug Enforcement Agency (DEA) and United States Department of Agriculture (USDA). The current threshold for total % THC by volume is 0.3%. Since the inception of the 2018 Farm Bill, and ensuing commercial legalization of Industrial Hemp, over 20% of United States Industrial Hemp crops have failed testing since 2018, with trends unchanged for the 2020 season (USDA, 2019).

In the United States 128,320 acres of hemp were reported cultivated in 2019 (USDA, 2020). With a fail rate of at least 20%, this equates to a minimum of 25,664 acres of failed crops. In 2020, 456,787 acres of Industrial hemp were cultivated in the United States, with a projected and unchanged fail rate of 20% by the USDA (USDA, 2020). This equates to 91,357 acres of failed hemp crops and a steady, maintained 20% fail rate of US crops since 2018.

Furthermore, equipment calibration, accuracy and sensitivity play a significant role in pass/fail rates. Without standardized testing equipment and procedures, results frequently vary from testing site to testing site, while using identical samples. This is causing significant issues and uncertainties as well as tremendous financial loss throughout the industry, including bankrupting companies.

The work herein, seeks to remove all issues and uncertainties surrounding % THC thresholds and testing variables by developing a true 0.000% THC Cannabis Sativa L. genotype. The introduction of this genotype will allow for cross breeding and further development of 0.000% THC cultivars, which will eliminate failed crop potentials, assure absolute lowest % THC levels at the plant genetic level (0.000%) and eliminate DEA and USDA concerns of % THC levels in retail/consumer products. Moreover, the development of the 0.000% THC genotype (and its ensuing cultivars) will allow for highest purity Cannabidiol-based (CBD-based) products, therefore eliminating FDA concerns and providing the safest and most compliant products to the consumer.

### Procedure Details

01) Protoplast development material was acquired via sampling Cannabis Sativa L. genotypes and determining the samples with highest efficiency to construct induction. Methods used include acquiring tissue samples from various stages of maturity/growth in order to obtain data on the stage(s) of highest efficiency. Samples were obtained from plants at 10 days growth from germination, 18 days growth from germination, 30 days growth from germination, and 60 days growth from germination. All plants were maintained on 18 hours on/6 hours off lighting schedule to ensure only vegetative growth. All plants and seedlings were cultivated indoors to maintain desirable environmental conditions and control. Temperatures remained constant at 77° F. during daylight schedule and 70° F. during night schedule. Relative humidity remained constant at 55% RH and carbon dioxide levels remained constant at 450 ppm.

02) Seeds of various Cannabis Sativa L genotypes were germinated in Rockwool substrate and trained to specific measures in order to produce multiple primordial leaf shoot sites. Primordial leaf shoots are the youngest/least mature plant structures known to maintain cell wall structures which can be most effective in protoplast and tissue culture development. Seedlings at 10 days germination were used as whole plant samples to obtain cellular material for protoplast development. Seedlings of this stage of growth are also known to provide higher efficiency rates in protoplast development and cell membrane removal.

03) Plants were irrigated using 0.8 ec/350 ppm nutrient solution after germination and 1.0 ec/500 ppm nutrient solution following 14 days post germination. Plants and seedlings remained under 6000k, T8 florescent lighting at no more than 600 μm/ft2 PPFD light intensities to maintain sample integrity.

04) Primordial leaf samples are handled using 4inch stainless steel forceps and cut with stainless steel sheers. Samples are removed approximately 1 inch from apical tips and placed in beaker containing 1.5% H2O2 solution. Excess growth is removed, leaving 0.25 inch-0.50 inch sample cuttings. Sheers and tweezers are rinsed in 10% sodium hypochlorite solution and rinsed in distilled H2O prior to each cutting and handling.

05) All samples are then washed and prepared in 1.5% H202 solution followed by a rinsing in distilled H2O to remove any contaminants and placed into sterile tubes.

06) Sterile tubes are then placed into chilled travel containers (35° F.-38° F.) and transferred to the laboratory.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

1. A synthetic DNA molecule comprising a nucleotide sequence encoding a gene editing agent and at least one guide RNA (gRNA) wherein the gRNA is complementary to a target nucleic acid sequence in a cannabis plant.

2. The synthetic DNA molecule of claim 1, wherein the gRNA is complementary to a nucleic acid sequence of a tetrahydrocannabinol (THC) gene.

3. The synthetic DNA molecule of claim 1, further comprising a sequence encoding a transactivating small RNA (tracrRNA).

4. The synthetic DNA molecule of claim 3, wherein the transactivating small RNA (tracrRNA) sequence is fused to the sequence encoding the guide RNA.

5. The synthetic DNA molecule of claim 1, wherein the nucleotide sequence encodes a first and second gRNA.

6. The synthetic DNA molecule of claim 5, wherein a first gRNA is complementary to a 5′ end of the THC gene and the second gRNA is complementary to a 3′ end of the THC gene.

7. The synthetic DNA molecule of claim 6, wherein the nucleic acid sequence between the 5′ gRNA target sequence and the 3′gRNA target sequence is excised.

8. The synthetic DNA molecule of claim 1, wherein the gene editing agent introduces deletions or mutations which inhibit expression of the THC gene.

9. The synthetic DNA molecule of claim 1, wherein the gRNA target sequence is in a THC gene regulatory region.

10. The synthetic DNA molecule of claim 1, wherein the gene-editing agent comprises CRISPR-associated nucleases, Argonaute family of endonucleases, clustered regularly interspaced short palindromic repeat (CRISPR) nucleases, zinc-finger nucleases (ZFNs), transcription activator-like effector nucleases (TALENs), meganucleases, endo- or exo-nucleases, or combinations thereof

11. An expression vector encoding the synthetic DNA molecule, wherein the synthetic DNA molecule comprises a nucleotide sequence encoding a gene editing agent and at least one guide RNA (gRNA) wherein the gRNA is complementary to a target nucleic acid sequence in a cannabis plant.

12. A host cell comprising a synthetic DNA molecule, wherein the synthetic DNA molecule comprises a nucleotide sequence encoding a gene editing agent and at least one guide RNA (gRNA) wherein the gRNA is complementary to a target nucleic acid sequence in a cannabis plant or an expression vector expressing the synthetic DNA.

13. The host cell of claim 12, wherein the cell is a Cannabis sativa cell.

14. A method of expressing a synthetic DNA molecule in a plant, comprising introducing into a host cell an expression vector encoding the synthetic DNA molecule in the host cell, wherein the synthetic DNA molecule comprises a nucleotide sequence encoding a gene editing agent and at least one guide RNA (gRNA) wherein the gRNA is complementary to a target nucleic acid sequence in a cannabis plant.

15. The method of claim 14, wherein the gRNA is complementary to a target nucleic acid sequence of a tetrahydrocannabinol (THC) gene.

16. The method of claim 15, wherein the THC is excised or inactivated.

17. A genetically-engineered plant or seeds thereof, wherein the plant does not express THC.

18. (canceled)


SEQUENCE LISTING (20 sequences)

SEQ ID NO: 1 - DNA, 24 bp, Artificial Sequence
  Description of Artificial Sequence Synthetic oligonucleotide
gaagaataagactacagtacatgg

SEQ ID NO: 2 - DNA, 24 bp, Artificial Sequence
  Description of Artificial Sequence Synthetic oligonucleotide
gaactttggtacactgctacctgg

SEQ ID NO: 3 - DNA, 20 bp, Artificial Sequence
  Description of Artificial Sequence Synthetic oligonucleotide
aattatggccttgcggctga

SEQ ID NO: 4 - DNA, 20 bp, Artificial Sequence
  Description of Artificial Sequence Synthetic oligonucleotide
accccaaatacgtgcttgtg

SEQ ID NO: 5 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 6 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 7 - protein, 6 aa, Artificial Sequence
  Description of Artificial Sequence Synthetic 6xHis tag
HHHHHH

SEQ ID NO: 8 - DNA, 21 bp, Artificial Sequence
  Description of Artificial Sequence Synthetic oligonucleotide
gaagaataagactacagtaca

SEQ ID NO: 9 - DNA, 21 bp, Artificial Sequence
  Description of Artificial Sequence Synthetic oligonucleotide
tgtactgtagtcttattcttc

SEQ ID NO: 10 - DNA, 21 bp, Artificial Sequence
  Description of Artificial Sequence Synthetic oligonucleotide
gaactttggtacactgctacc

SEQ ID NO: 11 - DNA, 21 bp, Artificial Sequence
  Description of Artificial Sequence Synthetic oligonucleotide
ggtagcagtgtaccaaagttc

SEQ ID NO: 12 - DNA, 29 bp, Artificial Sequence
  Description of Artificial Sequence Synthetic oligonucleotide
cttgcgccaccggggagtggtggggggtt

SEQ ID NO: 13 - DNA, 30 bp, Artificial Sequence
  Description of Artificial Sequence Synthetic oligonucleotide
cttgcgccgccgagggagtggtggggggtt

SEQ ID NO: 14 - DNA, 28 bp, Artificial Sequence
  Description of Artificial Sequence Synthetic oligonucleotide
cttgcgccgccggggagtggtgggggtt

SEQ ID NO: 15 - DNA, 28 bp, Artificial Sequence
  Description of Artificial Sequence Synthetic oligonucleotide
cttgccccaccgggagtggtggggggat

SEQ ID NO: 16 - DNA, 28 bp, Artificial Sequence
  Description of Artificial Sequence Synthetic oligonucleotide
cttgcgccgccgggagtggtggggggtt

SEQ ID NO: 17 - DNA, 27 bp, Artificial Sequence
  Description of Artificial Sequence Synthetic oligonucleotide
cttgcgccgccgggagtggtgggggtt

SEQ ID NO: 18 - DNA, 26 bp, Artificial Sequence
  Description of Artificial Sequence Synthetic oligonucleotide
cttgcgccaccgagtggtggggggtt

SEQ ID NO: 19 - DNA, 22 bp, Artificial Sequence
  Description of Artificial Sequence Synthetic oligonucleotide
cttgcgcgagtggtggggggtt

SEQ ID NO: 20 - DNA, 48 bp, Artificial Sequence
  Description of Artificial Sequence Synthetic oligonucleotide
aggatcaaaggtccatggagtgctgaagaagatcgggttttaactcgt